(19) United States
(12) Patent Application Publication
(10) Pub. No.: US 2002/0065503 A1
(43) Pub. Date: May 30, 2002

(54) Absorbent product

(76) Inventor: Ted Guidotti, Goteborg (SE)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 09/990,303

(22) Filed: Nov. 23, 2001

Related U.S. Application Data
(60) Provisional application No. 60/252,593, filed on Nov. 24, 2000.

(51) Int. Cl. A61F 13/15; A61F 13/20

(52) U.S. Cl. 604/389; 604/391

(57) ABSTRACT

The invention relates to an absorbent product, such as a diaper or an incontinence garment, comprising a casing, an outer side of the casing being distal from the body of the wearer in use of the garment and an inner side being proximal to the body of the wearer in use of the garment, said casing having a front part a rear part and a crotch part lying between the front and rear parts, and a fastening system for attaching opposing side portions of the front and rear parts to each other comprising at least two co-operating fastening elements, one of which functions as a landing zone for the other, characterised in that at least one element of the fastening system comprises means for making at least a portion thereof visible in the dark, in order to make it possible to adjust or exchange the diaper in the night, without turning on the light, and thereby disturbing the wearer's sleep more than necessary.

### TECHNICAL FIELD

The invention relates to an absorbent product, such as a diaper or an incontinence garment, comprising a casing, an outer side of the casing being distal from the body of the wearer in use of the garment and an inner side being proximal to the body of the wearer in use of the garment, said casing having a front part, a rear part and a crotch part lying between the front and rear parts, and a fastening system for attaching opposing side portions of the front and rear parts to each other, comprising at least two co-operating fastening elements, one of which functions as a landing zone for the other.

### BACKGROUND OF THE INVENTION

Today, several types of fastening systems for baby-diapers exist. There are tapes and tape landing zones, hook-and-loop systems and many more. The aim of the fastening system is to keep the diaper in place and maintain a good fit thereof to make sure that no leakage occurs. During the night, the fit of the diaper may have to be adjusted or the diaper may need to be exchanged when the user is sleeping, due to movements while sleeping or of other reasons. In those cases, because of difficulties to see the fastening system, the light has to be lit in order to be able to properly see the different parts of the fastening system when adjusting the diaper. When the light is lit, the user's sleep may be disturbed, and the user may wake up. If the user already is awake, it may be more difficult for him or her to get back to sleep, when it is not dark.

From U.S. Pat. No. 4,662,875 and U.S. Pat. No. 5,133,707 it is known to provide the fastening system with position indicators. However, none of these indicators are suitable for use in the dark.

Thus, there is a need for an absorbent product, which is constructed in a way that makes it possible to adjust its fastening system, without more than necessary disturb the wearer of the absorbent product, and which facilitates adjustment of the product in a dark or sparsely lit environment

U.S. Pat. No. 5,776,123 discloses an absorbent product having tactile position indicators. The purpose is to be able to adjust ones incontinence diaper or the like without seeing the fastening system. This document is mainly directed to incontinence diapers.

Accordingly, an object of the present invention is to provide an absorbent product, such as a diaper, in which the fastening system is easily adjustable in the dark.

### SUMMARY OF THE INVENTION

The object of the invention is accomplished by an absorbent product, such as a diaper or an incontinence garment, comprising a casing, an outer side of the casing being distal from the body of the wearer in use of the garment and an inner side being proximal to the body of the wearer in use of the garment, said casing having a front part, a rear part and a crotch part lying between the front and rear parts, and a fastening system for attaching opposing side portions of the front and rear parts to each other, comprising at least two co-operating fastening elements, one of which functions as a landing zone for the other, characterised in that at least one element of the fastening system comprises means for making at least a portion thereof visible in the dark.

In this way, the diaper can be exchanged and/or adjusted in the dark, without unnecessarily disturbing the person wearing the diaper. It is not necessary that the entire fastening system is visible in the dark. It is sufficient that a part of it is visible in the dark. Preferably, the co-operating fastening elements comprise at least one fastening tab and a finding zone, which functions as a receiving area for the fastening tab.

According to a first embodiment, at least the part of the fastening system comprising the landing zone comprises means for making at least a portion thereof visible in the dark.

Accordingly, another embodiment is an absorbent product, wherein both elements of the fastening system comprise means for making at least a portion thereof visible in the dark.

Still another embodiment is an absorbent product, wherein only the element adhering to the landing zone at least partly is visible in the dark. In this way, the tab, or the like, is visible. This embodiment may for example be practical when the entire side on winch the element comprising the landing zone is attached functions as a landing zone, such as for example is the case when that side is of a textile material, and the other element is a hook-element having the ability to adhere to such a textile material.

Yet another embodiment is an absorbent product, wherein the outer bound of the landing zone is visible in the dark. In tis way the fastening system, and especially the landing zone, is easily distinguished from the surrounding parts of the diaper. This may be done by applying lines or dashes, which are visible in the dark, surrounding rounding the fastening system.

Still another embodiment is an absorbent product, wherein at least one spot, preferably two spots, is applied to the outer side of the element adhering to the landing zone, and at least one spot, preferably two spots, is applied to the landing zone, whereby the spots essentially are applied in line with each other, and are visible in the dark. In this way, the two elements of the fastening system can be related to each other when adjusting the fastening system.

Yet another alternative is an absorbent product, wherein at least the element of the fastening system comprising the landing zone is manufactured of alight-emitting material or a material that is more visible in the dark than the surrounding parts.

In this way the fastening system is easily distinguished from the surrounding parts of the absorbent product.

Still another alternative is an absorbent product, wherein the means for making at least a portion of the landing zone visible in the dark is a fluorescent or phosphorescent colour, which makes the fastening system at least partly visible during the time period tat the colour emits light.

### BRIEF DESCRIPTION OF THE DRAWINGS

FIG. 1 discloses in a perspective view an absorbent product according to one embodiment of the invention.

FIGS. 2-4 Show alternative embodiments of the fastening system.

### DETAILED DESCRIPTION OF THE INVENTION

By an object being “visible in the dark” is meant that it is possible to distinguish the object from the surroundings even though it is essentially dark in the environment where the observer is present, i.e. the object has a lightness, which contrasts to the surrounding material. The term “essentially dark” does not necessarily indicate a totally dark environment, but rather an environment, which a person experiences as dark, as e.g. in a room with no light at night, but with some light emitting through curtain, blinds or through a chink of the door.

The definition of “the landing zone” in the context of this invention also comprises a material sheet, such as a part of the backing sheet, functioning as the area to which a fastening tab, or the like, of the fastening system, may adhere.

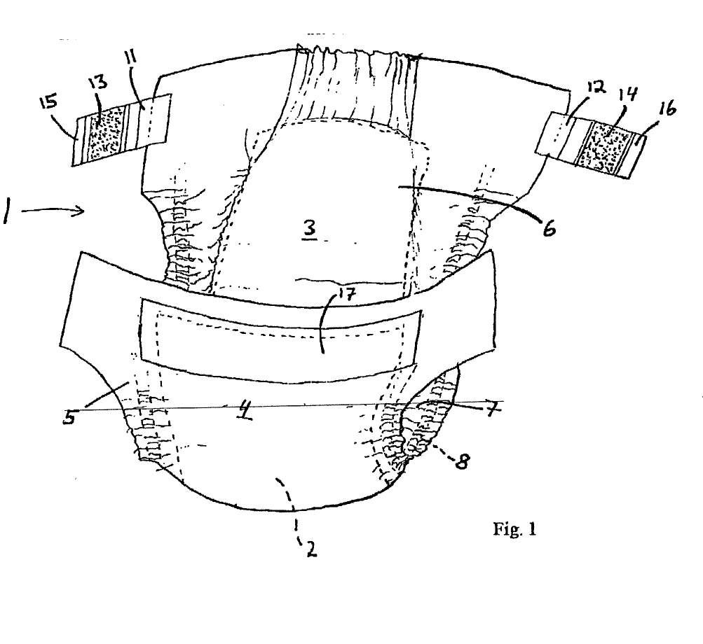

FIG. 1 discloses a diaper 1. As is conventional in this field of the art, the diaper comprises an absorbent body 2, being enclosed between a liquid-permeable top sheet 3 and a liquid-impermeable big sheet 4, these sheets being fastened to each other in portions thereof reaching beyond the absorbent body. The diaper has a front part 5, a rear part 6 and an intermediate crotch part 7, the longitudinal direction of the diaper extends from the rear to the front edge thereof. The diaper also comprises leg elastics, for example one or several elastic threads or bands disposed between the top sheet and the backing sheet along the sides of the diaper, at least in the crotch part thereof, and attached to the top sheet and/or backing sheet in a stretched state. The top sheet 3 and the backing sheet 4 constitutes a casing for the absorbent body 2 and the side portions of the front 5 and rear part 6 of the diaper 1 are laterally projecting from the front and rear parts in relation to the lateral extension of the casing in the crotch part 7 of the diaper.

Further, the absorbent diaper comprises an adjustable fastening system, comprising at least two cooperating fastening elements (13, 14, 17), one of which functions as a landing zone (17) for the other (13, 14). The fastening elements (13, 14, 17) attaches opposing side portions of the front and rear parts to each other in order to give the diaper a pants-like configuration. The extension of the element functioning as a landing zone (17) in the side portions of the front part 5 of the diaper is such that if the opposing fastening elements (13, 14) are attached to the respective outer end of the element 17, the opposing side portions of the rear and front parts are slightly overlapping.

The absorbent product may for example be of a “hour-glass”-shape, wherein the “ears” of the time-glass constitutes the side portions of the absorbent product. The fastening element functioning as a landing zone 17 is then applied close to the waist-line of the outer side of either the front or rear part of the product. The other element (13, 14) is preferably a pair of fastener tabs (11, 12) attached to the side edges close to the waist-line of either the rear or the front part of the product. Out-side the element (13) 14) a grip tab (15, 16) is provided, in order to be gripped by hand when adjusting the fastening system. In a preferred embodiment the landing zone (17) is in the front part of the product and the fastener tabs (11, 12) are attached to the rear part.

According to the first embodiment, the element of the fastening system comprising the landing zone (17) is manufactured of a material making it distinguishable in the dark, such, as a light-emitting material, or a material being whiter than the surrounding materials.

In a preferred embodiment the element functioning as a landing zone 17 is a landing zone for adhesive tape tabs or for hook and loop tabs (13, 14). Preferably, the area of the element functioning as a landing zone is larger than the area of the opposing element, in order to give a possibility to adjust the length of the waistline of the diaper when applied on a wearer.

The element functioning as a landing zone, does not have to be a specific landing zone, applied on the backing sheet or on the front sheet material. The material of the backing (or the front sheet) may also be constructed of a material to which a tab can adhere, such as a backing sheet made of a suitable textile material. Thereby, the definition of “the landing zone” in the context of the invention also comprises a material sheet, such as a backing sheet or a part of the backing sheet, functioning as the element to which a tab, or the like, of the fastening system may adhere.

The liquid-permeable top sheet 3 is made of a soft skin-friendly material. Examples of suitable materials are different types of non-woven. Other material that can be used are perforated plastic films, plastic nets or knitted, crotched or woven textile materials or combinations and laminates of the above mentioned types of material. The plastic material can be a thermoplastic material, for example polyethylene (PE). The non-woven material can be formed of natural fibres, such as cellulose or cotton fibres, but ban alternatively consist of synthetic fibres, such as PE, polypropylene (PP), polyurethane (PU), a polyester, nylon or regenerated cellulose, or a mix of different fibres. All materials used for top sheets in absorbent sanitary garments can be used for the top sheet 3 and the above mentioned materials are only given as examples.

The liquid-impermeable backing sheet 4 consists of a flexible material, preferably a thin plastic film of PE, PP or a polyester but could also consist of a laminate of a liquid-permeable material, such as a non-woven, and a liquid-impermeable material, such as e.g a plastic film. All materials that are used for liquid-impermeable backing sheets are conceivable. The backing sheet can advantageously be air-permeable.

The absorbent body 2 is preferably formed of cellulose fibres but also other natural materials, such as cotton fibres or peat can be used. Alternatively, absorbent synthetic fibres or a mixture of natural and synthetic fibres may be used. The absorbent body can also comprise a super-absorbent, i.e. a polymer having the capacity to sorb liquid to an amount several times larger than its own weight. The absorbent body can also contain form stabilizing and/or liquid dispersing components and also bonding agents for holding together short fibres and particles to a continuous unit. Furthermore, the absorbent body can contain more than one layer of absorbent material.

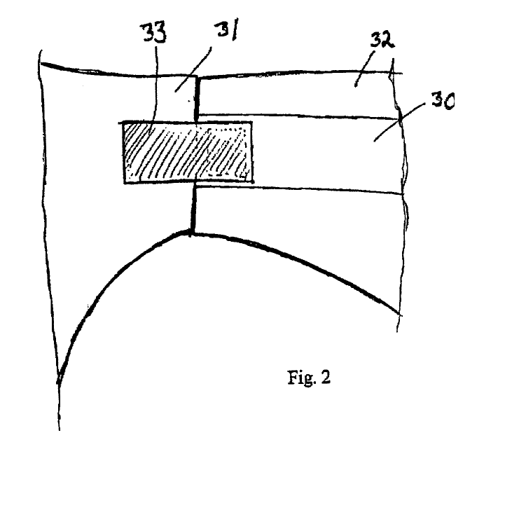

FIG. 2 shows the fastening system of another embodiment of the invention. A tab (33) attached to the side portion (31) of the front or rear part of a diaper is adhered to a landing zone (30) attached to the side portion (32) of the opposite side of the diaper (front or rear part). The top part of the tab (33), shaded in picture, is manufactured of a material that is visible in the dark, thereby making it possible to see and adjust the tab in the dark.

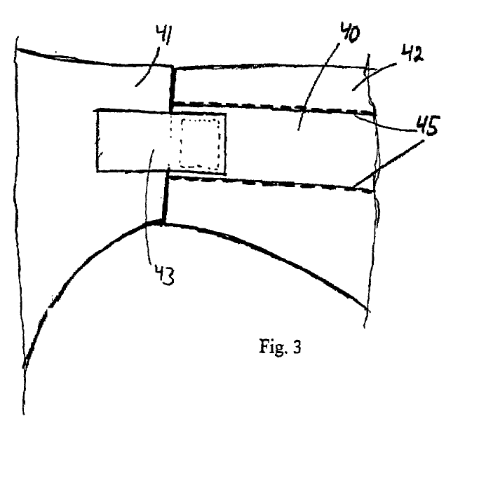

FIG. 3 shows the fastening system of yet another embodiment of the invention. A tab (43) attached to the side portion (41) of the front or rear part of a diaper is adhered to landing zone (40) attached to the side portion (42) of the opposing part of the diaper (front or rear part). The landing zone is made visible in the dark by a dashed line (45) surrounding the landing zone. The dashed line is made of a colour that is visible in the dark. The line may also be continuous or made in any other way that makes the fastening system or part of it distinguishable in the dark.

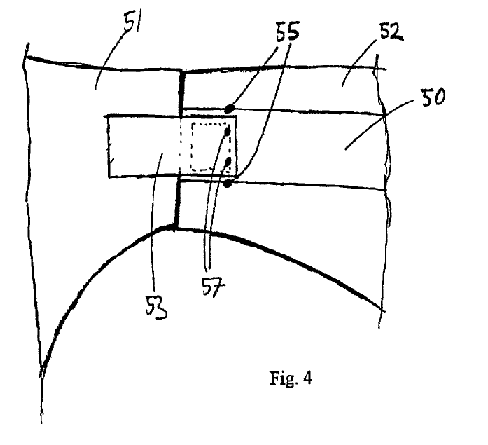

FIG. 4 shows the fastening system of still another embodiment of the invention. A tab (53) attached to the side portion (51) of the front or rear part of a diaper is adhered to a landing zone (50) attached to the side portion (52) of the opposite side of the diaper (front or rear part). Two spots (57) that are visible in the dark are applied on the top part of the tab (53), and two other spots (55) that is visible in the dark are applied on the landing zone (50), preferably “in line” with the spots on the tab (57). In this way the fastening system can be adjusted in the dark, the different elements of the fastening system be related to each other.

In the embodiments described above, a tab and a tab landing zone has been described. However, the invention applies to any kind of fastening system. Further, the landing zone must not necessarily be constructed in the way described above. Many different sizes and constructions are fully possible within the scope of the invention. Moreover, other combinations of spots, lines, marks etc. may be used, as well as combinations of the described embodiments, in order to make the fastening system or part of it visible in the dark. For example, some kind of darkly visible marking may occur, like spots in a lateral row along the landing zone, which indicates the position on the landing zone. In case the entire backing sheet functions as a landing zone, darkly visible markings on the backing sheet indicating where to fasten the fastening tab may occur, in order to provide a good fit.

In order to provide a fastening system that at least partly is visible in the dark, the material of which the different parts of the system are produced, may be luminous by itself, of it may comprise luminous substances or colour, or luminous substances may be applied on the surface of the material. In this way, the material has the ability to emit light in the dark, which light previously has been absorb ed

Any suitable luminous substances, phosphorogenic or flourescent substances and colours known in the art may be used, or other substances that are able to absorb and store light (energy) and emit it in the dark. The substances being luminous should be able to emit light during a time interval of at least a few hours, preferably ten hours and most preferably more than twenty-four hour, in order to make it functional during different conditions. These conditions include when the user is sleeping at night, but also when the user is sleeping in a darkroom during longer periods than a night, such as e.g. in a hospital or likewise.

Moreover, the fastening system may at least partly be whiter or lighter than the surrounding parts, thereby making it easier to be observed in the dark, or at least make it easier to see when it is not completely dark. In this way, the fastening system can be distinguished when it is dark, and therefore also be adjusted. Materials or colours that are suitable in this respect include those having good reflection characteristics or fluorescence characteristics, thereby providing an easily distinguishable surface, line, spot or the like, with only a small amount of light provided. Examples of such materials can be found in reflexes used for traffic safety, as on clothing, cars or road signs. Further, the fastening system, or part of it, may also be of any other colour than white, which makes the coloured part more visible in the dark than the surrounding part.

The invention is suitable for baby diapers, but a fastening system that is visible in the dark may of course also be practical in many other absorbent p ducts, such as incontinence garments, when it is an advantage to perform the adjustment or exchange of the diaper in the dark.

The fastening system that are used i the absorbent product according to the invention may be of several different kinds, such as an adhesive tape and a tape landing zone, a hook and loop type of fastening system, as well as other kinds.

The description illustrates how the invention maybe performed. However, the description is not intended to limit the scope of the invention, and modifications of the exemplified embodiments are fully possible. For example, the two elements of the fastening system may both be applied to the front part as well as to the rear part. Further, the absorbent product may be of different kinds, such as diapers and incontinence garments. The construction of the absorbent product may include an absorbent product, or may provide for an insert. The element of the fastening system, which adheres to the landing zone part, may also be attached to the inside of the ears, instead of the fastener tab.

1. Absorbent product (1), such as a diaper or an incontinence garment, comprising a casing (3, 4), an outer side of the casing being distal from the body of the wearer in use of the garment and an inner side being proximal the body of the wearer in use of the garment, said casing having a front part (5), a rear part (6) and a crotch part (7) lying between the front and rear parts, an a fastening system for attaching opposing side portions of the front and rear parts to each other, comprising at least two co-operating fastening elements, one a which functions as a landing zone (17, 30, 40, 50) for the other (13, 33, 43, 53) characterised in that at least one element of the fastening system comprises means for making at least a portion thereof visible in the dark.

2. Absorbent product according to claim 1, wherein the element of the fastening system/comprising the landing zone (17, 30, 40, 50) comprises means for making at least a portion thereof visible in the dark.

3. Absorbent product according to claim 2, wherein both element of the fastening system comprise means for making at least a portion thereof visible in the dark.

4. Absorbent product according to claim 1 or 2, wherein the element adhering to the landing zone at least partly is visible in the dark (33).

5. Absorbent product according to claim 1, wherein the outer bound (45) of the landing zone is visible in he dark.

6. Absorbent product according to claim 1 or 2, wherein at least one spot (57), preferably two spots, is applied to the top side of the element adhering to the landing zone, and at least one spot (55), preferably two spots, is applied of the landing zone, whereby the spots essentially are applied in line with each other, and are visible in the dark.

7. Absorbent product according to anyone of the preceding claims, wherein the means or making at least a portion of the fastening system visible in the dark is manufactured of a light-emitting material or a material that is more visible in the dark than the surrounding parts.

8. Absorbent product according to anyone of claims 1-6, wherein the means for making at least a portion of the fastening system visible in the is a fluorescent or phosphorescent colour.
